(19) United States
(12) Patent Application Publication
(10) Pub. No.: US 2007/0016207 A1
(43) Pub. Date: Jan. 18, 2007

(54) Skull pin apparatus

(76) Inventor: Endelman Ken, Sacramento, CA (US)

Correspondence Address: GREENBERG TRAURIG LLP, 2450 COLORADO AVENUE, SUITE 400E, SANTA MONICA, CA, 90404, US

(21) Appl. No.: 11/174,809

(22) Filed: Jul. 1, 2005

(51) Int. Cl. A61B 17/58

(52) U.S. Cl. 606/72

(57) ABSTRACT

A skull pin for use in a halo type skull fixation device is disclosed for maintaining a patient's skull in a predetermined position. The skull pin has an elongated substantially cylindrical pin body that includes a threaded portion and a distal end portion aligned along a central axis, and a separate skull engaging tip member. The tip member is mounted on the distal end portion of the elongated body. The tip member has a cup shaped socket rotatably receiving therein the distal end portion of the body and a pointed end opposite the socket axially aligned with the distal end portion of the body. A low friction material is disposed between the cup shaped socket and the distal end portion of the pin permitting rotation of the pin body relative to the tip about the central pin axis to minimize tearing of a patient's skin when the pins in the fixation device are periodically adjusted during use.

### BACKGROUND OF THE INVENTION

1. Field of the Invention

This invention generally relates to halo type head fixation devices and more particularly to a skull pin for use in a head fixation device.

2. Description of Related Art

Today, many types of head fixation devices are available. Some devices have screws which are threaded into a patient's skull to maintain a precise alignment and others have a conical shaped pin which is pressed into the skull of the patient. Typically there are three or four such pins used to maintain position.

One of the problems with such devices is that readjustment of the pins is usually needed after initial placement to compensate for changes in the overall structure over a period of time in which the fixation device is required to be worn, which can be several months. When adjustments are made, the threaded pin is rotated into the patient's skull. This causes the surrounding skin of the scalp to twist and abrade which is extremely uncomfortable to the patient.

### SUMMARY OF THE INVENTION

Against this backdrop the present invention has been designed. A preferred embodiment of the present invention is a skull pin adapted to be supported in a halo type skull fixation device in which the skull pin has a floating tip so that the tip does not tend to rotate when the skull pin is tightened against the skull of the patient. The skull pin includes an elongated threaded body and a separate tip. The tip has a cup shaped socket. The cup shaped socket of the floating tip captures a distal end portion of the skull pin body. Inside and between the cup shaped tip and the distal end portion of the skull pin body is a low friction substance that permits the tip and the body to be rotated independent of one another while maintaining axial alignment of the tip and body. One embodiment of the distal end portion of the skull pin body is preferably coated with a low friction film such as a polytetrafluroethylene (PTFE) film which acts as a lubricant to permit the body to rotate in the fixation device while the tip remains stationary in a patient's skull. Another embodiment of the skull pin has a low friction sleeve inside the cup shaped tip over the distal end portion of the threaded body. Another embodiment has a lubricating fluid

These and other features, advantages and objects of the invention will become more apparent from a reading of the following detailed description when taken in conjunction with the accompanying drawings.

### BRIEF DESCRIPTION OF THE DRAWINGS

FIG. 1 is a side view of a halo type skull fixation device utilizing a skull pin in accordance with an embodiment of the present invention.

FIG. 2 is a longitudinal cross sectional view of a skull pin in accordance with an embodiment of the present invention for use in the device shown in FIG. 1.

FIG. 3 is a separate longitudinal cross sectional view of a skull pin in accordance with another embodiment of the present invention.

FIG. 4 is a longitudinal cross sectional view of a skull pin in accordance with an alternative embodiment of the present invention.

FIG. 5 is a longitudinal cross sectional view of a skull pin in accordance with another alternative embodiment of the present invention.

### DETAILED DESCRIPTION OF THE INVENTION

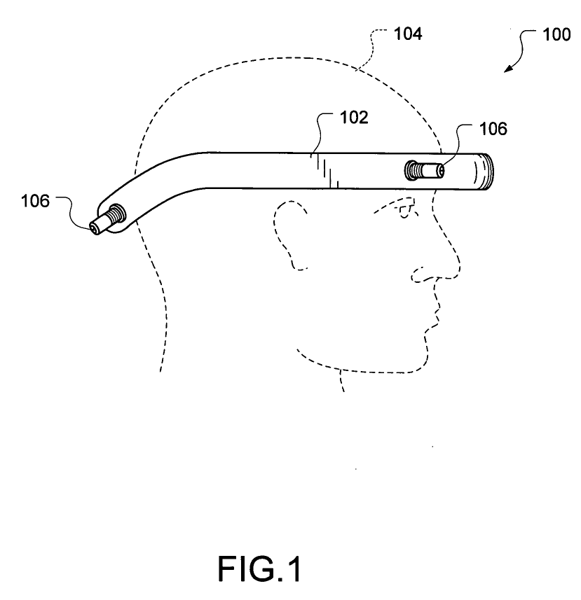

A side view of a halo type skull fixation device 100 positioned about a patient's skull 104 (shown in phantom) is shown in FIG. 1. The fixation device 100 has a curved, generally U shaped frame 102 and includes a plurality of threaded bores receiving therethrough skull pins 106. These skull pins 106 are positioned in the device frame 102 such that the skull 104 of the patient is held in a specific position. The frame 102 is then fastened to a structure that rides on the patient's shoulders, chest and back to maintain relatively rigid alignment between the patient's torso and head while upper or cervical vertebra fractures are permitted to heal.

Periodically, it is necessary for adjustments of the fixation device 100 to be made during use. The skull pins 106 become loose or too tight against the skull 104 as the skull shrinks or expands over time. During such adjustments, the skull pins must be turned. In the present disclosure, the pins 106 have a separate tip that permits the body of the pin 106 to be turned without turning the tip, thus minimizing tearing of a patient's skin around the pins 106 and thus minimizing pain typically associated with such adjustments.

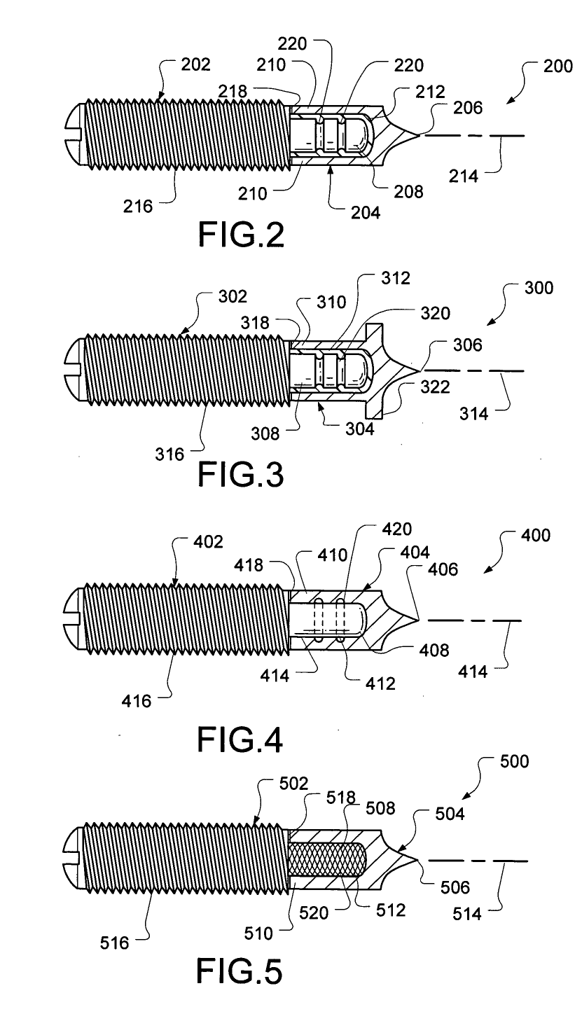

Several particular embodiments of skull pins 106 for use in halo device 100 are shown in the subsequent FIGS. 2, 3, 4 and 5. Turning now to FIG. 2, a longitudinal cross sectional view of one embodiment of a skull pin is shown. This skull pin 200 has an elongated, substantially cylindrical body 202 and a separate skull engaging tip member 204 with a pointed end 206 mounted on the distal end portion 208 of the elongated body 202. The tip member 204 has a cup shaped socket 210 rotatably receiving therein the distal end portion 208 of the body 202 and a pointed end 206 opposite the socket 210 axially aligned with the distal end portion 208 of the body 202. The skull pin 200 includes a low friction material 212 disposed between the cup shaped socket 210 and the distal end portion 208 of the pin body 202 permitting rotation of the pin body 202 relative to the tip 204 about the central pin axis 214.

The cylindrical body 202 has a threaded portion 216 aligned with the distal end portion 208, which has an outer diameter less than that of the threaded portion 216, thereby forming a shoulder 218 between the two portions of the body 202. The threaded portion 216 is sized to be threaded through the halo device frame 102 in a conventional manner. A lock nut (not shown) is typically also threaded onto the threaded portion 216 to lock the pin 200 in position in the frame 102.

The distal end portion 208 of the pin 200 preferably has a rounded semi-spherical end and a pair of circular peripheral grooves 220. However, other configurations of the end of the distal end portion may be utilized. The end may be flat or tapered to match the bottom of the cup shaped socket of the tip 204. In this embodiment 200, the peripheral grooves 220 receive and preferably are filled with the low friction material 212. In addition, there is preferably a thin layer of the low friction material 212 sandwiched between the shoulder 218 and the open end of the tip 204 so as to minimize friction between these two surfaces. In this embodiment 200, the low friction material 212 may be a polymeric material such as polytetrafluoroethylene (PTFE) or other polymer that is formed in place over and on the distal end 208 prior to assembly of the tip 204 to the body 202. Alternatively the material 212 may be a solid body cap separately pressed onto the distal end 208. The pointed end 206 of the tip 204 preferably has a concave tapered cross section so that only a small portion actually penetrates a patient's skull 104.

Turning now to FIG. 3, a longitudinal cross sectional view of another embodiment of a skull pin 106 is shown. The skull pin 300, like pin 200, has an elongated, substantially cylindrical body 302 and a separate skull engaging tip member 304 with a pointed end 306 mounted on the distal end portion 308 of the elongated body 302. The tip member 304 has a cup shaped socket 310 rotatably receiving therein the distal end portion 308 of the body 302 and a pointed end 306 opposite the socket 310 axially aligned with the distal end portion 308 of the body 302. The skull pin 300 again includes layer of a low friction material 312 disposed between the cup shaped socket 310 and the distal end portion 308 of the pin body 302 permitting rotation of the pin body 302 relative to the tip 304 about the central pin axis 314.

The cylindrical body 302 has a threaded portion 316 aligned with the distal end portion 308, which has an outer diameter less than that of the threaded portion 316, thereby forming a shoulder 318 between the two portions of the body 302. The threaded portion 316 is sized to be threaded through the halo device frame 102 in a conventional manner. A lock nut (not shown) is typically also threaded onto the threaded portion 316 to lock the pin 300 in position in the frame 102.

The distal end portion 308 of the pin body 302 preferably has a rounded semi-spherical end and a pair of circular peripheral grooves 320. However, other configurations of the end of the distal end portion may be utilized. The end may be flat or tapered to match the bottom of the cup shaped socket of the tip 304. In this embodiment 300, the peripheral grooves 320 receive and preferably are filled with the low friction material 312. In addition, there is preferably a thin layer of the low friction material 312 sandwiched between the shoulder 318 and the open end of the tip 304 so as to minimize friction between these two surfaces. In this embodiment 300, the low friction material 312 may also be a polymeric material such as polytetrafluoroethylene (PTFE) or other polymer that is formed in place over and on the distal end 308 prior to assembly of the tip 304 to the body 302. Alternatively the material 312 may be a solid body cap separately pressed onto the distal end 308. The pointed end 306 of the tip 304 preferably has a concave tapered cross section so that only a small portion actually penetrates a patient's skull 104. To enhance this limited penetration, tip 304 has a circular flange 322 around the point 306.

Turning now to FIG. 4, a longitudinal cross sectional view of one embodiment of a skull pin 106 is shown. The skull pin 400 has an elongated, substantially cylindrical body 402 and a separate skull engaging tip member 404 with a pointed end 406 mounted on the distal end portion 408 of the elongated body 402. The tip member 404 has a cup shaped socket 410 rotatably receiving therein the distal end portion 408 of the body 402 and a pointed end 406 opposite the socket 410 axially aligned with the distal end portion 408 of the body 402. The skull pin 400 includes a low friction material 412 disposed between the cup shaped socket 410 and the distal end portion 408 of the pin body 402 permitting rotation of the pin body 402 relative to the tip 404 about the central pin axis 414.

The cylindrical body 402 has a threaded portion 416 aligned with the distal end portion 408, which has an outer diameter less than that of the threaded portion 416, thereby forming a shoulder 418 between the two portions of the body 402. The threaded portion 416 is sized to be threaded through the halo device frame 102 in a conventional manner. A lock nut (not shown) is typically also threaded onto the threaded portion 416 to lock the pin 400 in position in the frame 102.

In this embodiment 400, the inner wall of the socket 410 has a pair of circular peripheral grooves 420. In this embodiment 400, the peripheral grooves 420 receive and preferably are filled with the low friction material 412, which in this embodiment is a low friction polymeric fluid. In addition, there is preferably a thin gap preferably filled with the low friction material 412 sandwiched between the shoulder 418 and the open end of the tip 404 so as to minimize friction between these two surfaces. In this embodiment 400, the low friction material 412 may be a polymeric material such as a polytetrafluoroethylene (PTFE) grease or other polymer fluid placed on the distal end 408 prior to assembly of the tip 404 to the body 402.

Turning now to FIG. 5, a longitudinal cross sectional view of a still further embodiment of a skull pin 106 is shown. The skull pin 500 has an elongated, substantially cylindrical body 502 and a separate skull engaging tip member 504 with a pointed end 506 mounted on the distal end portion 508 of the elongated body 502. The tip member 504 has a cup shaped socket 510 rotatably receiving therein the distal end portion 508 of the body 502 and a pointed end 506 opposite the socket 510 axially aligned with the distal end portion 508 of the body 502. The skull pin 500 includes a low friction material 512 disposed between the cup shaped socket 510 and the distal end portion 508 of the pin body 502 permitting rotation of the pin body 502 relative to the tip 504 about the central pin axis 514.

The cylindrical body 502 has a threaded portion 516 aligned with the distal end portion 508, which has an outer diameter less than that of the threaded portion 516, thereby forming a shoulder 518 between the two portions of the body 502. The threaded portion 516 is sized to be threaded through the halo device frame 102 in a conventional manner. A lock nut (not shown) is typically also threaded onto the threaded portion 516 to lock the pin 500 in position in the frame 102.

In this embodiment 500, the distal end portion 508 of the body 502 has helical grooves 520 formed therein to distribute the low friction polymeric fluid 512. In addition, there is preferably a thin gap preferably filled with of the low friction material 512 sandwiched between the shoulder 518 and the open end of the tip 504 so as to minimize friction between these two surfaces. In this embodiment 500, the low friction material 512 may be a polymeric material such as a polytetrafluoroethylene (PTFE) grease or other polymer fluid placed on the distal end 508 prior to assembly of the tip 504 to the body 502.

Various other configurations and alternatives are envisioned. For example, in embodiments 200, 400 and 500, a peripheral flange as in embodiment 300 may be provided to minimize skull penetration. Although a flat screwdriver slot is shown in the proximal ends of the pins shown in FIGS. 2-5, other drive arrangements may be provided. For example, either a male or female hex socket or square socket head, or Phillips screwdriver slotted head configuration may be utilized instead of the flat blade head as shown. The material of the pin tips 204, 304, 404, and 504 is preferably titanium, although other materials that are medically compatible may alternatively be used. The pin bodies may also be made of titanium or alternatively a stainless steel material.

It will be clear that the present invention is well adapted to attain the ends and advantages mentioned as well as those inherent therein. While a presently preferred embodiment has been described for purposes of this disclosure, numerous changes may be made which will readily suggest themselves to those skilled in the art and which are encompassed in the spirit of the invention as set forth in the following claims.

What is claimed is:

1. A skull pin for use in a halo for maintaining a patient's skull in a predetermined position, the skull pin comprising:
an elongated substantially cylindrical pin body having a threaded portion and a distal end portion having a central axis;
a separate skull engaging tip member mounted on the distal end portion of the elongated body, the tip member having a cup shaped socket rotatably receiving therein the distal end portion of the body and a pointed end opposite the socket axially aligned with the distal end portion of the body; and
a low friction material disposed between the cup shaped socket and the distal end portion of the pin permitting rotation of the pin body relative to the tip about the central pin axis.

2. The skull pin according to claim 1 wherein the low friction material is a layer of polymer lining the cup shaped socket.

3. The skull pin according to claim 1 wherein the low friction material is a layer of polymer fastened to the distal end portion of the pin body.

4. The skull pin according to claim 1 wherein the low friction material is a lubricant fluid.

5. The skull pin according to claim 1 wherein the low friction material is polytetrafluoroethylene.

6. The skull pin according to claim 1 wherein the distal end portion of the pin body has at least one peripheral groove to retain the low friction material on the distal end portion of the pin body.

7. The skull pin according to claim 1 wherein the cup shaped socket has an inside circular groove receiving a portion of the low friction material therein.

8. The skull pin according to claim 1 wherein the skull engaging tip member has a peripheral shoulder projecting outward from the socket around the pointed end.

9. The skull pin according to claim 1 wherein the distal end portion of the pin body has helical scoring to permit the low friction material to flow between the distal end portion of the pin and the socket during insertion of the distal end portion into the socket.

10. The skull pin according to claim 3 wherein the layer of polymer extends between a shoulder on the threaded portion and the socket of the tip.

11. A halo skull fixation device for securing a patient's skull in a predetermined position comprising:
a generally curved halo frame sized to fit around a patient's head; and
a plurality of threaded skull pins spaced around and threaded through the frame for engaging the patient's head to maintain the patient's skull in position, wherein each skull pin comprises:
an elongated substantially cylindrical pin body having a central axis and having a threaded portion and a distal end portion;
a separate skull engaging tip member mounted on the distal end portion of the elongated body, the tip member having a cup shaped socket rotatably receiving therein the distal end portion of the body and a pointed end opposite the socket axially aligned with the distal end portion of the body; and
a low friction material disposed between the cup shaped socket and the distal end portion of the pin permitting rotation of the pin body relative to the tip about the central pin axis.

12. The device according to claim 11 wherein the low friction material is a layer of polymer lining the cup shaped socket.

13. The device according to claim 11 wherein the low friction material is a layer of polymer fastened to the distal end portion of the pin body.

14. The device according to claim 11 wherein the low friction material is a lubricant fluid.

15. The device according to claim 11 wherein the low friction material is polytetrafluoroethylene.

16. The device according to claim 11 wherein the distal end portion of the pin body has at least one peripheral groove to retain the low friction material on the distal end portion of the pin body.

17. The device according to claim 11 wherein the cup shaped socket has an inside circular groove receiving a portion of the low friction material therein.

18. The device according to claim 11 wherein the skull engaging tip member has a peripheral shoulder projecting outward from the socket around the pointed end.

19. The device according to claim 11 wherein the distal end portion of the pin body has helical scoring to permit the low friction material to flow between the distal end portion of the pin and the socket during insertion of the distal end portion into the socket.

20. The device according to claim 13 wherein the layer of polymer extends between a shoulder on the threaded portion and the socket of the tip.
